(19) United States
(12) Patent Application Publication
(10) Pub. No.: US 2021/0349169 A1
(43) Pub. Date: Nov. 11, 2021

(54) METHODS TO FACILITATE AND GUIDE DATA ANALYSIS USING MRµTEXTURE AND METHOD OF APPLICATION OF MRµTEXTURE TO DIAGNOSIS OF COVID-19 AND OTHER MULTI-ORGAN DISEASES

(71) Applicant: BIOPROTONICS, INC., Santa Barbara, CA (US)

(72) Inventors: Kristin James, Santa Barbara, CA (US); Timothy W. James, Santa Barbara, CA (US)

(21) Appl. No.: 17/308,266

(22) Filed: May 5, 2021

Related U.S. Application Data
(60) Provisional application No. 63/020,344, filed on May 5, 2020.

(51) Int. Cl. G01R 33/58; G01R 33/48; A61B 5/00; A61B 5/055

(52) U.S. Cl. CPC G01R 33/58; G01R 33/4818; A61B 5/4504; A61B 5/055; A61B 5/7257; A61B 5/4381; A61B 5/0042; A61B 5/425; A61B 5/4244

(57) ABSTRACT

A method for calibration of the MRμTexture method is presented wherein a plurality of model datasets representing a continuum of structures with a continuum of biomarker values is generated by morphing data of a 2D structure or 3D structure of a first known disease state to a 2D structure or a 3D structure of a second known disease state. MRμTexture is applied in silico to extract a simulation data set of texture prevalence for a selected one of a plurality of intermediate morphed conditions corresponding to the plurality of model datasets.

### REFERENCES TO RELATED APPLICATIONS

This application claims priority of U.S. provisional application 63/020,344 filed on May 5, 2020 entitled METHODS TO FACILITATE AND GUIDE DATA ANALYSIS USING MRμTEXTURE AND METHOD OF APPLICATION OF MRμTEXTURE TO DIAGNOSIS OF COVID-19 AND OTHER MULTI-ORGAN DISEASES having a common assignee as the present application, the disclosure of which is incorporated here by reference. Data analysis methods are described to facilitate interpretation of the data output from the magnetic-resonance-based diagnostic tool described in U.S. Pat. Nos. 9,366,738, 9,664,759, 10,061,003, 10,330,763, 10,215,827, and U.S. application Ser. Nos. 16/450,361 and 16/68,976, all having a common assignee with the present invention, the disclosures of which are incorporated herein by reference.

### BACKGROUND

### Field

This application relates generally to a magnetic-resonance-based diagnostic method, referred to in this document as MRμTexture (for Magnetic Resonance Microtexture), as disclosed in the citations of REFERENCES TO RELATED APPLICATIONS, enabling sensitive and accurate measurement of the microstructural state of, and changes in, biologic tissue textures and correlation of major chemical constituents of the tissue with specific spatial frequencies in the textural data output was disclosed in this series of patents and, more particularly to a method for calibration of MRμTexture using high information content ground truth data.

### Related References

Disease happens quietly. Changes begin at the very smallest levels of the anatomy, affecting the microscopic structure of the biologic tissue of which organs are composed. A huge unmet need in healthcare is the ability to assess these very fine changes, before they lead to irreversible pathology accumulation. The list of diseases for which accurate measure of tissue changes would enable sensitive diagnosis is extensive. It includes bone disease, bone degradation from cancer treatment as disclosed in Novel magnetic resonance technique for characterizing mesoscale structure of trabecular bone, C. Nguyen et al., Royal Society Open Science, rsos.royalsocietypublishing.org/Sep. 24, 2018, diseases marked by fibrotic development, such as liver disease, lung disease, kidney disease, and cardiac disease, neurologic diseases and conditions including the various forms of dementia, multiple sclerosis, cerebrovascular disease, and tumor formation in a range of cancers such as prostate disease as disclosed in MR method for measuring microscopic histologic soft tissue textures, G. Sonn et al., Magnetic Resonance in Medicine, 2021; 00:1-12 Hence, it provides a powerful tool to apply to the task of unraveling disease etiology, diagnosing, and monitoring progression in a disease such as COVID-19, a hallmark of which is its multi-organ attack, with hugely varied presentation and course.

Currently, the only direct way to measure microscopic changes in biologic tissue texture is biopsy, an invasive procedure, fraught with sampling errors—biopsies often miss their intended target, such as a small tumor in early-stage development. The invasiveness of biopsy limits its use in some organs, limits the number of samples obtainable from any given organ, and limits the ability to repeat studies for longitudinal tracking of disease and therapy response. Also, application of biopsy to an immune-compromised patient is contra-indicated. However, though MR imaging is the diagnostic of choice in a wide range of diseases due to its ability to non-invasively provide tunable tissue contrast to highlight variations in the anatomy, spatial resolution in MR imaging is limited by blurring caused by patient motion. This makes it impossible to image the microscopic changes in tissue texture that signal disease onset, or that would enable tracking disease progress. Even using cardiac and respiratory gating schemes or real-time motion correction, and with a compliant patient, resolution is not high enough to measure microscopic tissue texture. Certain MR contrast mechanisms such as DWI (Diffusion-Weighted-Imaging) look at signals affected by the microscopic texture of biologic tissue, however the signals obtained by use of this method are indirect, and hence not unique—different underlying cellular states can be responsible for a specific output signal—there is not a one-to-one correspondence. As a result of this inability to measure biologic tissue texture at high resolution, noninvasively (in vivo), much nascent pathology goes undetected because the microscopic biologic tissue changes attendant with disease onset and progression are outside the resolution capability of current clinical imaging techniques. Not only does this affect outcomes, but the inability to target subject participants early enough in disease course seriously hampers therapy development efforts.

### SUMMARY

A method for calibration of the MRμTexture method is presented.

A plurality of model datasets representing a continuum of structures with a continuum of biomarker values is generated by morphing data of a 2D structure or 3D structure of a first known disease state to a 2D structure or a 3D structure of a second known disease state. MRμTexture is applied in silico to extract a simulation data set of texture prevalence for a selected one of a plurality of intermediate morphed conditions corresponding to the plurality of model datasets.

The features, functions, and advantages that have been discussed may be achieved independently in various implementations or may be combined in other implementations further details of which can be seen with reference to the following description and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described herein are for illustration purposes only and are not intended to limit the scope of the present disclosure in any way.

FIG. 1A is an example biologic structure using Trabecular bone structure;

FIG. 1B is an idealized bone structure created from the biologic structure of FIG. 1A;

FIG. 2 is a flowchart of an example calibration sequence;

FIG. 3 is a representation of in silico simulation of loss of bone strut thickness and resultant microtexture spectra as obtained with MRμTexture;

FIG. 4A is an image of normal lung tissue;

FIG. 4B is an image of COVID-19 disease showing thickened alveoli walls compared to FIG. 4A;

FIG. 5 is a Myelin-stained section of human cortex (bar is 500 micrometers) showing the characteristic texture in this tissue;

FIG. 6 is representative set of images from one control (case 1146: A 5 PT, B 5 HG), one MCI case (case 489: C 5 PT, D 5 HG), and one AD case disclosed in “Cerebral Cortex” August 2011; 21:1870-1878, doi:10.1093/cercor/bhq264, Advance Access publication Jan. 14, 2011 (case 176: E 5 PT, F 5 HG) with region PT illustrated on the left and HG from the same case shown on the right wherein wide spacing of minicolumns can be seen in control PT (A) with narrower minicolumns in HG (B) while thinning of minicolumns is found in the MCI PT (C) more similar in width to HG (D) and narrow and disrupted minicolumns are seen in AD (E and F);

FIG. 7A shows a of a 4 mm thick section radical prostatectomy specimen;

FIG. 7B shows the prostate gland histology from a first side of the specimen; and,

FIG. 7C shows the prostate gland histology from a second side of the specimen. through

### DETAILED DESCRIPTION

Table of terms:

| | |
|---|---|
| VOI | Volume of Interest/sampling volume |
| k-space. | is an array of numbers representing spatial |
| | frequencies in the MR image |
| k-value | One spatial frequency |
| MR | Magnetic resonance |
| MRμ Texture | Magnetic Resonance Microtexture |
| In silico | performed on computer or via computer |
| | simulation |
| MCI | Mild Cognitive Impairment |
| Chemical shift | is the resonant frequency of a nucleus relative |
| | to a standard (e.g., water) in a magnetic field |

The aim of the methods described herein is to maximize the information that can be extracted from the output data acquired using the MR-based diagnostic tool, MRμTexture. Further, these methods will guide targeting of specific data for acquisition to maximize diagnostic information. The implementations herein disclose methods for calibration of the MRμTexture for diagnosing specific diseases based on analysis of the output data. The ability of MRμTexture to measure pathology in many, highly varied organ tissues enables a direct correlation of a pathology measurement between organs. The fact that tissue state is assessed by the same measurement method enables direct comparison of the pathology state of the tissue across organs, greatly facilitating correlation of the measurements and assessment of pathology state across the anatomy.

The previously unmet challenge of obtaining in vivo, noninvasive, clinically robust, high resolution MR measure of tissue texture can be met using the MRμTexture technology.

The MRμTexture technology, as disclosed, uses the fact that magnetic resonance scanners acquire data in diffraction space (k-space) to allow design of an MR data acquisition sequence that enables motion immune (very high resolution) acquisition of tissue texture measurement data. Diffraction space is comprised of a matrix of signal at each of the spatial frequencies that contribute to an image—this spectrum being obtained by Fourier analysis.

To generate a high-resolution MR image, a very large data set is required starting at k=0 and continuing up to the highest frequency Fourier component present in the image. This can be understood with reference to a spectrum analyzer, such as the sound analyzers built into some stereo systems. An audio spectrum analyzer breaks an acoustic waveform into a spectrum of signal strength vs. audio frequency of the sound frequencies that contribute to the audio signal. Diffraction space is simply a plot of MR image data that shows the relative contribution of each of the spatial frequencies that comprise an MR image. Applying a Fourier transform to this frequency-space data yields the MR image. But, to form an image, the relative intensity of a continuous range of spatial frequencies must be measured in the anatomy to be imaged, all measurements needing to be in phase, from 0 (the DC-value) up through the highest spatial frequency desired in forming the image. The smaller the features in the anatomy, the greater the range of spatial frequency intensities that must be recorded to resolve them in the image. The problem is that the large range of spatial frequency intensity data needed to form an image makes for a very large data set, especially as spatially resolved data must be acquired across the entire organ being imaged. As a result, the SNR (Signal to Noise Ratio) for each individual data point in the acquisition is low. This problem of low SNR is exacerbated by the fact that signal amplitude varies inversely with spatial frequency-higher resolution features generally emit lower signals. Therefore, multiple excitations are required for signal averaging to boost SNR. But over the time needed to acquire all this data patients are moving, and the image is blurring, so that very fine features will not be resolvable. In MR imaging, the need to acquire data across a large range of spatial frequencies in each excitation, and across a large spatial extent, results in motion-limited tissue texture resolution.

By contrast, MRμTexture enables a very high-resolution, clinically robust measure of tissue texture by focusing on measuring the signal intensity of only those spatial frequencies pertinent to the targeted pathology, and specifically not trying to build up a conventional image. MRμTexture acquires data at a specific k-value, or small set of k-values, within a single excitation. Motion within the excitation does not affect this measure because, once excited, the tissue signal is not affected by motion. Measurements of signal intensity at additional k-values are achieved by repeating this small set of k-values acquisition across other excitations but now coherence across this set of k-values is not important—it is simply the relative signal intensity across the acquired set of k-values that is needed. The only requirement on patient motion using MRμTexture is that the sampling volume remain within a similar region of tissue during the time the various spatial frequency intensities are measured to characterize the tissue—a much more lenient requirement than the spatial phase coherence that is needed for imaging. The MRμTexture diagnostic provides a new quantitative MR measure that enables in vivo tissue texture measure anywhere in the anatomy, allowing mapping of data across organs, with the ability to repeat the measure as often as a patient is in the scanner, to track pathology.

Obtaining MRμTexture data is accomplished at a summary level by transmitting a first RF pulse with a first gradient chosen for first slice selection; transmitting a second RF pulse with application of a second gradient chosen for slice selective refocusing in a region defined by an intersection of the first slice and a second slice; encoding a specific k-value with a selected gradient pulse; transmitting a third RF pulse with a third gradient activated, said third gradient adapted for slice selective refocusing, defining a region defined by the intersection of the first and second slices and a third slice selection to define a volume of interest (VOI); turning off all gradients; and, recording multiple samples of an RF signal encoded with the specific k-value in a single excitation.

The basic MRμTexture method may be supplemented by applying a non-zero magnitude gradient as a time-dependent phase-encode determining a trajectory through k-space while recording samples of at sequential a sequence of k-values across a neighborhood of k-values defined by height and pulse width of the non-zero magnitude gradient, the sequence of k-values being a subset of k-values required to make an image; and post processing samples at a combination of sequential k values, recorded within a time span while the non-zero magnitude gradient is applied.

Additionally, applying a contrast mechanism enhancing the contrast between the component tissue types in a multiphase biologic sample being measured may also be employed. Also by taking the Fourier Transform of the acquired signal data for each k-encode, wherein the signal data recorded is indicative of the spatial power density at that point in k-space; and, evaluating each peak in the NMR spectrum whereby the relative contribution to texture of tissue in the VOI at a k-value the chemical species in the sample may be determined.

An alternate approach for defining the VOI in the MRμTexture method accomplished by transmitting a first RF pulse with a first gradient chosen for first slice selection in a specimen; transmitting a second RF pulse with application of a second gradient chosen for slice selective refocusing in a region defined by an intersection of the first slice and a second slice defining a rod within the specimen; applying an encoding gradient pulse to induce phase wrap to create a spatial encode for a specific k-value and orientation; applying a low non-zero magnitude gradient having a first magnitude acting as a time dependent phase encode to produce a time varying trajectory through 3D k-space of k-value encodes; simultaneously recording multiple sequential samples of the NMR RF signal at a sequence of k-values across a neighborhood proximate the specific k-value defined by height and pulse width of the non-zero magnitude gradient in a single excitation; setting a first receiver bandwidth to delineate a length of a VOI within the rod during the data sampling; and post processing the samples at the sequence of k values, recorded within a time span while the non-zero magnitude gradient is applied, to characterize the textural features of the specimen in the VOL.

Measurement accuracy (i.e., the ability to identify and differentiate specific tissue types) for the MRμTexture method relies on accurate determination of the transfer function between the underlying morphologic tissue texture features targeted in a measurement and the MRμTexture output data set. The term “transfer function” is used in this context to mean that, for a given targeted tissue sample (tissue type), the output from the MRμTexture method is known and predetermined. As MRμTexture provides a direct measure of texture, the output textural wavelength spectrum contains all of this information. However, biologic tissues are often relatively complex in morphology. Interpretation of the output data from a diagnostic method such as MRμTexture to uniquely characterize the microtexture of the targeted tissue requires detailed determination of this transfer function between underlying texture and data output. Determination of the calibration/transfer function linking the MRμTexture data output with the underlying tissue texture/pathology state enables sensitive/accurate determination of the targeted tissue morphology. Establishing the transfer function is defined herein as calibrating the MRμTexture method.

In silico modelling of biologic tissue textures combined with in silico modeling of acquisition of MRμTexture data from these structures is used to develop accurate calibration of the method, i.e. accurate determination of the transfer function linking the modeled tissue textures with the resultant MRμTexture data output as described in the detailed examples below.

In general, modeling is often used towards understanding and simplifying systems under study. There are various ways to build models of tissue structures that reproduce tissue morphology pertinent to disease. These models are either created mathematically or derived from biologic data available from the tissue under study. These models (simulations) are then varied as described elsewhere in this disclosure.

A few possible sources of tissue morphology data are—both optical and Scanning Electron Microscopy, stained 2-dimensional histopathology/histomorphometry images, 3-dimensional MR-microscopy (long term MR imaging of excised tissue), and microCT data of both bone and soft tissue. All these data translate into 2-dimensional or 3-dimensional maps of signal intensity vs. location which provide the basis for models of tissue morphology.

The mapping of tissue morphology to simulate a 2D or 3D spatial map of the structure of various types of biologic tissue includes an assigned signal intensity for each point in the 2D or 3D spatial model. In addition to the morphologic data provided by the various forms of microscopy/histology, the simulated signal values chosen can be based on modeling tissue properties, including chemical species, T2 decay, T1, proton density, etc. which, as they are spatially resolved, enable determination of the morphology of chemical constituents.

Using these techniques, a plurality of model datasets representing a continuum of structures with a continuum of biomarker values is generated by morphing data of a 2D structure of a first known disease state to a 2D structure of a second known disease state.

In-silico simulation of the MRμTexture method signals from an in silico model of tissue (a 2D or 3D model with “signal” values for each location in space) is performed applying MRmTexture in silico to extracting a simulation data set of texture prevalence for each of the plurality of intermediate morphed conditions corresponding to the plurality of model datasets. by methods including:

1—Fourier analysis of the 2D or 3D in silico model and selecting the Fourier coefficients along the axis corresponding to the desired analysis direction in the VOL. This approach provides a Fourier series of k-encoded simulated MRμTexture method signals.

2—Simulation of the signal for a single k-encode by first summing the signal values for all points on the one or two axes (for 2D and 3D models respectively) orthogonal to the analysis direction in the VOI for each point along the analysis direction. This generates a 1D signal intensity vs. position along the analysis direction of the VOL. This 1D array is then multiplied by a complex sinusoid with a wavelength corresponding to the desired k-encode—the complex sum of the points in this product array provides the simulated MRμTexture method signal.

To achieve desired calibration of the MRμTexture method, one approach is to develop models of tissue structures pertinent in specific diseases, starting with very simple models of tissue structures, and adding complexity to modeling the advancement of tissue changes known from histology to occur in specific organ pathology development. This complexity may take the form of increasing randomness of tissue texture as disease progresses, but the aim is to simplify the tissue model sufficiently to enable identification of specific tissue texture features with specific features in the data output, such as high intensity signals from certain structural wavelengths.

With each iteration of the tissue texture model, correlation is made between the modeled tissue features and the data output from the in silico modeled MRμTexture data acquisition. This enables the development of a transfer function linking tissue morphology with MRμTexture data output.

This correlation can be accomplished by starting with a very simplified model of the tissue and adding complexity, observing the corresponding changes in the MRμTexture output spatial frequency spectrum with each texture change. Alternatively, changes in the spatial frequency spectrum can be made, and observation made of the corresponding changes in the targeted tissue textures—this correspondence achieved through use of the reverse Fourier Transform applied to a spatially coherent set of k values (which is easily achieved in silico).

As part of developing these correlations, the tissue features can be varied singly or with multiple changes made at once. Texture spacing, element thickness, texture anisotropy, textural variability/texture heterogeneity, chemical composition of texture, varied tissue contrast, are a few of the methods of varying the modeled tissue features to mimic pathology development.

These methods can be used to calibrate the MRμTexture method for application to determining pathology advancement and disease etiology attendant with COVID-19 progression by measuring changes in specific textural features, such as vasculature spacing, density, and randomness, neuron-bundle degradation as measured by increasing structural randomness, fibrotic development in liver and lung pathology progression. These texture feature changes are then used to identify and correlate pathology advancement in multiple organs. Again, the starting point for correlating MR μTexture is histology data either 2D or 3D across a range of illness starting with healthy tissue.

Another use of the above-described in silico modeling, data acquisition and analysis of tissue textures, with attendant library building towards determination of the MRμTexture transfer function, is for fast reading of biopsy samples acquired during surgery or as part of other diagnostic procedures. Tissue biopsies acquired as a step in disease diagnosis commonly require, transfer to slides, cutting, fixation, and staining to enable biopsy read. Another possible approach is to apply the MRμTexture method to direct read of tissue biopsies, significantly speeding up this process, resulting in a much faster assessment of tissue pathology. Calibration of this application is achieved similarly to the above-described determination of the MRμTexture transfer function using tissue modeling, in silico MRμTexture data acquisition from the tissue, and correlation with the actual MRμTexture data output. Standard biopsy reads are used as ground truth for correlation to develop an accurate MRμTexture transfer function. The combination of in silico modeling of tissue texture morphology with correlating MRμTexture in silico data acquisition to build up a library of biopsy tissue texture measure vs. MRμTexture data output enables much faster read of tissue biopsies, obviating the need for the detailed process of sample staining and slide production and slide reading.

One method to accurately determine this transfer function, or MRμTexture method calibration, is through use of high information content ground truth data, such as tissue histology. This data can be used for calibration of the MRμTexture output data. In this approach, the high-resolution ground truth measure of ex vivo tissue textures, such as tissue histology” is correlated with the data output obtained by MRμTexture textural analysis from the same or similar tissue.

High information content ex vivo ground truth data is available from 2D histology slice images acquired from the targeted tissue type/disease state and stained to reveal the desired pathologic tissue texture components of interest. This histology can be obtained from ex vivo or postmortem tissue slices, from the literature, or from histology atlases. Additionally, optical microscopy, or a new, microCT-based 3D soft tissue histology technique may prove useful in certain tissues to reveal tissue changes that can provide ground truth for calibration of the MRμTexture signal. This ability for 3D ground truth is currently under development by O. L. Katsamenis at University of Southampton, UK. 3DμCT (Katsamenis et al., X-ray Micro-Computed Tomography for Nondestructive Three-Dimensional (3D) X-ray Histology, The American Journal of Pathology, Volume 189, Number 8, August 2019)

The basic methods disclosed herein are targeted towards accurate determination of the transfer function between the underlying tissue pathology, which is reflective of disease stage, and the data output from applying the MRμTexture diagnostic measurement to this targeted tissue to measure pathology stage. Determination of the transfer function between diagnostic method—MRμTexture—and the targeted tissue pathology is equivalent to calibration of the MRμTexture tissue texture measurement method. To effect this calibration of MRμTexture the following steps are used: 1) in silico modeling of tissue texture, based on ground truth histopathology, and variation of this model to mimic changes in texture resulting from pathology development, again using histopathology as the ground truth for tissue texture pathology development, 2) in silico application of MRμTexture data acquisition to this modeled tissue to acquire output data representative of the modeled pathology stage, and 3) precise correlation between specific features in the varied histopathology data and the MRμTexture data output, using this ongoing model variation to accurately and sensitively reveal the correlation between specific tissue texture features, and specific features on the MRμTexture output spectrum/data matrix. Further, the pattern-recognition capability of AI/machine learning analysis techniques can be used to facilitate extraction of diagnostic biomarkers from the data acquired from these modeled tissues.

As discussed previously, obtaining a 3-dimensional, or 2-dimensional, image of the targeted tissue structure in vivo, noninvasively, is fraught due to patient motion causing image blurring, hence reducing image resolution. However, calibration as disclosed herein may be accomplished using a combination of very high-resolution histology imaging of ex vivo tissue 1) to enable development of useful 2-dimensional and 3-dimensional models of these specific tissue structures under study and their pathology changes with disease development and 2) for accurate understanding of the transfer function that connects the underlying microscopic textures to the MRμTexture output.

Developing a highly specific correlation between the output data from the MRμTexture method and the underlying measured biologic tissue texture in healthy or diseased tissue is accomplished by using the histology images as the basis of knowledge of a 3D structural representation of a tissue region to be characterized. Then, in silico, the components of this structure are varied and observations made of the resultant changes in the in silico-acquired MRμTexture output data/spectrum from this modelled tissue. Each textural component can be varied—for instance, trabecular bone thickness and trabecular bone spacing, or cortical neuron bundle spacing and randomness,—and the effect this variation has on the MRμTexture data spectrum observed.

This will enable building up of libraries of specific textural features and the specific MRμTexture output spectral (or other data format) features they are linked with. These library entries can be simple correspondence between a single textural feature, such as fibrotic density, and MRμTexture output data, or can be generated by varying multiple textural characteristics in synchrony.

As an example using cancellous bone as the targeted tissue, and measurement of degeneration of the target tissue from cancer therapy, age, time in space, etc. is desired. As with most biologic tissue textures, cancellous bone has a relatively high degree of textural variability—variations in TbTh (Trabecular Thickness), TbSp (Trabecular Spacing) from location to location, as well as tissue anisotropy associated with gravitational loading. The directly causative feature of bone degradation is thinning of the trabecular elements (TbTh), which leads to sudden discontinuities in TbSp as these elements thin to the point of breaking. However, in terms of these changes in trabecular thickness the most salient tissue change occurs with the breaking of these trabecular element—tracking trabecular element thinning requires a very high sensitivity measure of tissue change. Ability to accurately quantify the continuous changes in TbTh that occur prior to breaking would provide an extremely sensitive measure of bone degradation. Because of its immunity to patient motion, MRμTexture can provide the sensitive measures of these structures to determine transfer function. This calibration of the MRμTexture output, is difficult due to the very fine changes in overall structure represented by trabecular thinning. By comparison, trabecular spacing provides a much stronger component of the MRμTexture data output so is easier to extract from the output data than is the signal from trabecular thinning. Further, the textural frequencies that comprise trabecular thickness fall at very high k-value, much higher than those from TbSp. As the Fourier coefficient of a textural component decreases in amplitude with increasing k-value, the component reflecting TbTh exhibits low SNR, and hence is harder to discern amongst the other textural spectral features in the data output.

Towards sensitive/robust determination of the transfer function between the microscopic bone texture and the MRμTexture data output/spectral features, two methods can be applied:

1) Develop a highly simplified in silico model of the tissue (in this case cancellous bone) and vary specific microstructural elements in silico, tracking the resultant changes in the MRμTexture output spectrum through in silico data acquisition from the modelled structure, or
2) Use histology of the targeted tissue texture 2D pathology staining, 3D microCT, or 2D optical microscopy, for instance, to generate a textural starting point (healthy tissue) and an end point (disease) to provide information on which characteristics of biologic tissue reflect disease most sensitively. Then, acquire data in silico from these 2D or 3D structure models. Starting with healthy tissue (or some lower level of pathology) vary targeted textural elements and acquire data from the modelled tissue in silico to track changes in the output data/spectrum that reflect the varied textural elements, and hence are indicative of disease progression. Using this method, one can morph one stage of disease into another and derive output spectra for intermediate stages of disease.

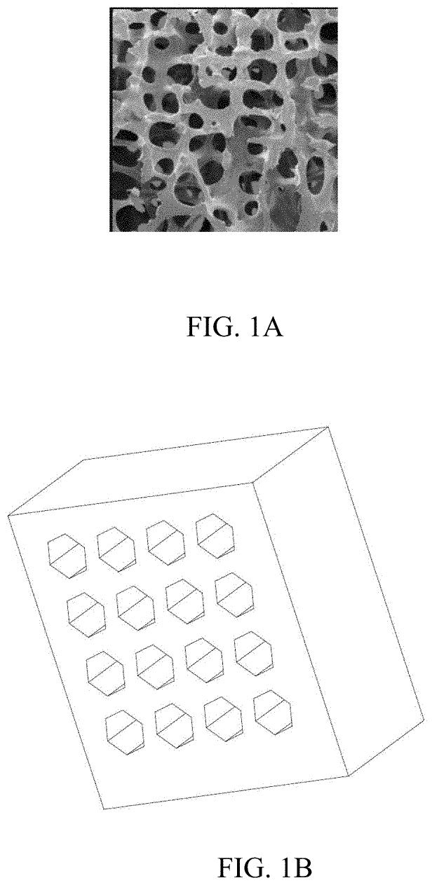

As an example of the in silico model approach, for bone, the simplest idealized microtexture could be a rectilinear system of equally-spaced elements in 3 orthogonal directions, with a single element repeat spacing and single element thickness at the start. (FIG. 1A shows an SEM image of a biologic tissue structure, and FIG. 1B shows a highly simplified structure of trabecular bone obtained by modelling.) Varying this simplified structure in silico by varying trabecular element thickness and spacing, and standard deviation and anisotropy of these measures, adding complexity in stages, while acquiring MRμTexture data in silico at each stage, enables clear correlation between specific textural elements and the associated MRμTexture data output/spectrum. This correlation is then extended, via study of histology, to train the MRμTexture method such that its output data can be correlated with tissue pathology—i.e. disease stage.

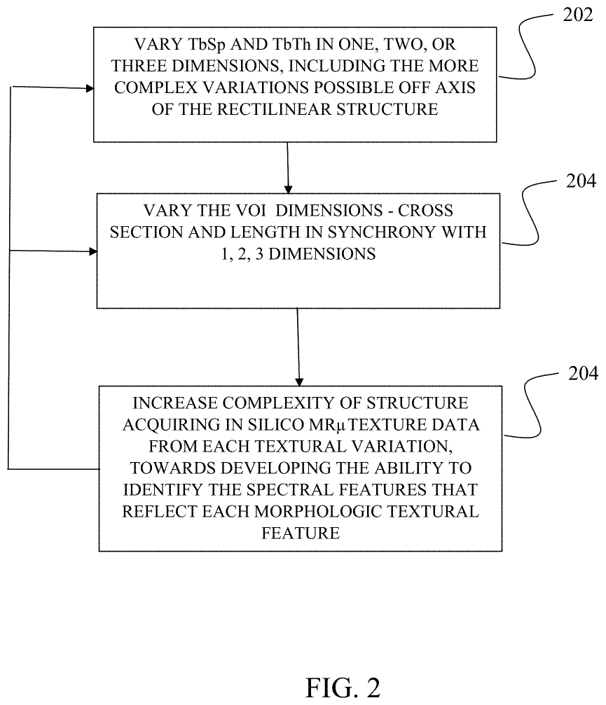

Toward identifying specific features in the MRμTexture data output with specific textural features in the targeted tissue the following steps as shown in FIG. 2 can be taken:
Vary TbSp and TbTh in one, two, or three dimensions, including the more complex variations possible off axis of the rectilinear structure, step 202.
Vary the VOI (Volume of Interest=sampling volume) dimensions-cross section and length in synchrony with 1, 2, 3 dimensions, step 204.
Increase complexity of structure acquiring in silico MRμTexture data from each textural variation, towards developing the ability to identify the spectral features that reflect each morphologic textural feature, step 206.

An alternative method would be to do this in reverse. Rather than varying the textural features and observing the effect on the MRμTexture in silico spectrum acquired, vary the MRμTexture spectrum and observe the change on the micro-texture.

The aim here is to use in silico modeling and varying of structures/output data to build up a library enabling correspondence between MRμTexture output data and specific changes in tissue textures. This then can be extrapolated to correlation between MRμTexture data output and pathology advancement.

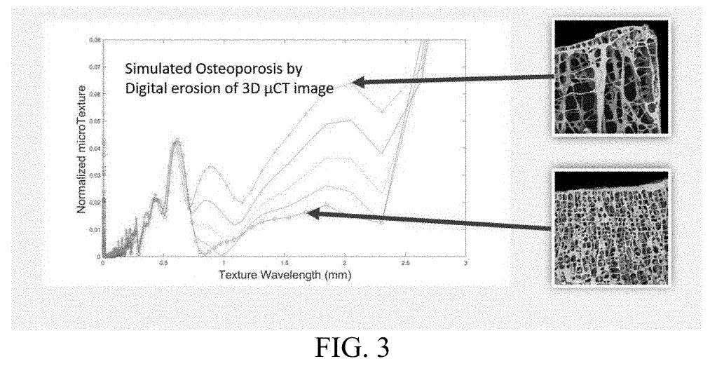

As an example of the histology approach, FIG. 3 demonstrates application of this process in trabecular bone, starting with a biological structure and varying it in silico. First, a high-resolution 3D microCT dataset was acquired from an ex vivo vertebral body and used as a starting point. In silico thinning of the trabecular elements in steps mimicked pathology advancement. At each thinning step, MRμTexture data was acquired in silico. The gradual change in the MRμTexture data spectra with each step can readily be seen. The series of spectra clearly show a stepped response, changes in the structure occurring most noticeably at longer wavelengths.

This continuous change in the spectrum in response to the continuous thinning of the bone will eventually lead to the elements disappearing altogether and hence a more drastic change in the MRμTexture output spectrum.

Correlation of the variation in output spectrum with the in silico modeled textural change from in silico bone thinning would enable determination of the spectral features that are indicative of trabecular element thickness.

Continuing with this example, start with bone using a very regular 3D structure. Modify it incrementally—spacing of elements and thickness; isotropy; variability—and watch the MR-μTexture output data/spectrum change.

Many diseases have histology, in vivo or ex vivo: this can be the start with healthy tissue structures varied towards pathology. Record changes in the MRμTexture signal. This method enables determination of the tissue texture variation of pathologic signals and say what texture change has occurred.

Signal components can be combined to yield a complex structural signal.

Various structural wavelength regions of the MRμTexture spectrum can be ratioed or combined and compared in other ways towards development of a biomarker.

Simple structures can be varied to see how the MRμTexture output data/spectrum changes.

MR microscopy, or any other technique that provides ground-truth information on tissue texture and its progressive variation with disease progress can be used as a starting point for in silico modeling and data acquisition.

Ex vivo measures enable much higher resolution of the tissue textures underlying disease towards defining various stages of tissue pathology.

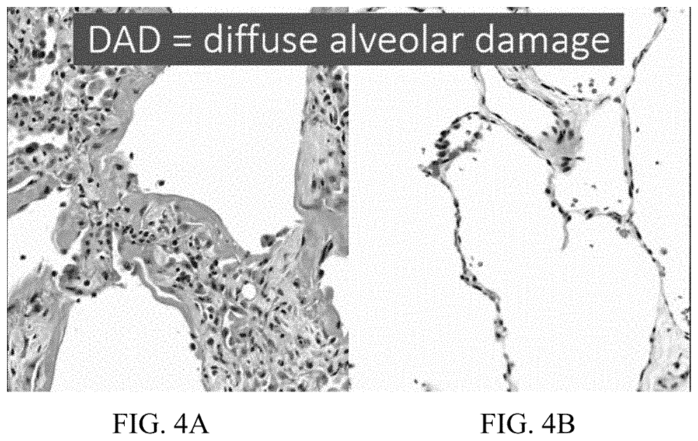

The development of lung fibrosis is associated with thickening of the alveoli walls, a healthy tissue texture being modified in stages by disease advancement. FIGS. 4A and 4B are an example of the different textures in normal lung tissue vs. COVID-19 lung tissue. Alveoli wall thickening alters the tissue texture but to the first order leaves the primary spatial wavelength (inverse k-value), a repeating pattern of the ˜200 μm diameter alveoli, unchanged. But the changes in wall thickness of the alveoli that occur in synchrony with disease advancement modify the spectrum of spatial frequencies (k-values) in the MRμTexture output data similarly to the effect of the thinning of trabecular elements in bone with advancing disease. With the caveat that, in bone the trabecular elements thin with advancing pathology whereas, in lung disease development, the boundaries of the alveolar wall are observed to thicken with disease progression. In either case, the basic analysis method remains the same—observation of modelling of tissue texture based on histological data and varying this model incrementally to match the changes observed, also with histology, in tissue texture with disease onset and progression, allows correlation between the MRμTexture output spectrum and the simplified tissue models obtained from histology. as outlined above in the bone case can be used to correlate the MRμTexture spectra with advancing pathology. Further association with histology would provide the basis for training AI algorithms to identify the sentinel texture sizes to target as a clinical tool for identifying stages of development of this tissue structure.

This can be done using 2D histology and models, as well as 3D histology and models.

An associated application would be to use MRμTexture to measure pathologic changes in blood vessels, to discern whether a low patient oxygenation might be due to constricted blood vessels rather than alveolar thickening/clogging. Histologic vasculature textural data from can be used to develop ability to discern in what ways the MRμTexture output spectrum changes as, for instance, angiogenic vasculature forms in response to tumor growth—angiogenic vasculature is characterized by high density, random microvessel development in the tumor regions. Again, basing the form of development of angiogenic vasculature on the histologic record, a model of healthy vasculature can be varied incrementally to model the development of angiogenic vasculature. This variation is then correlated with the incremental changes observed in the MRμTexture output data/spectrum to enable association of specific features in the spectrum of k values with specific changes in the diseased tissue texture. Again, either the model of the microvessel changes can be varied to mimic pathology development, and the changes in the MRμTexture spectrum observed, or changes in the MRμTexture data spectrum can be introduced, and observation of the tissue texture obtained from Fourier transforming the spectrum observed to enable correlation of the two measures.

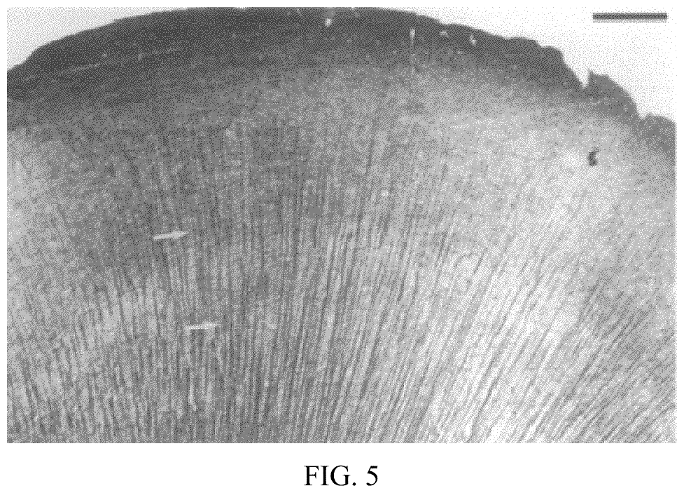

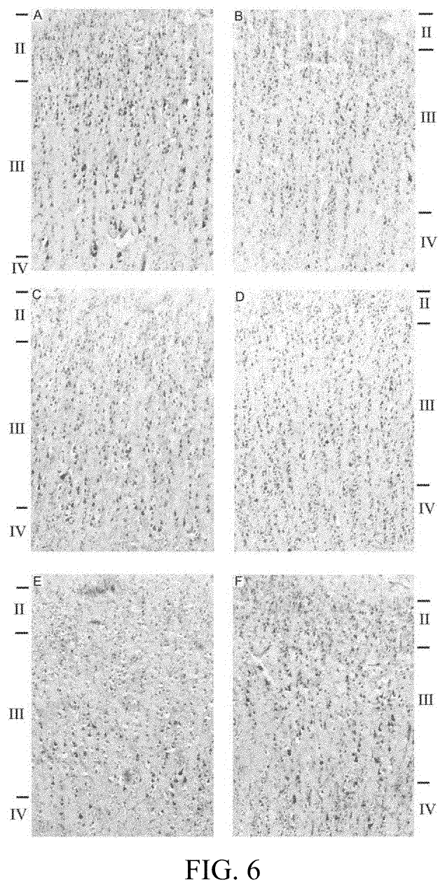

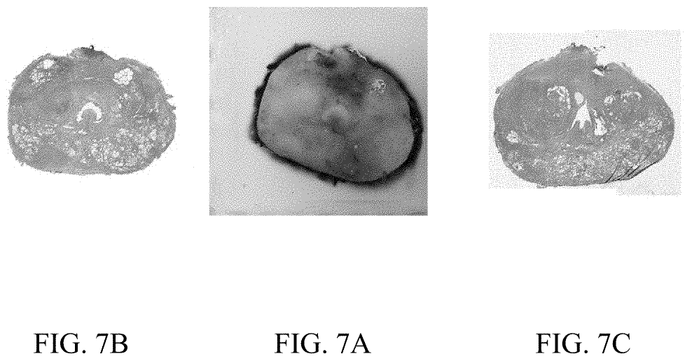

This same approach can be taken in other tissue systems, for instance in neuropathology. The attached histology image in FIG. 6 is of the myelinated bunches of neurons that traverse the cortex in columnar formation in healthy brain. In progression of various diseases, such as dementia, this ordered structure degrades over time as the myelin strips from the columns and the cells lose their ordered state, becoming spatially scrambled. The loss of order is shown in FIG. 5. A method to determine the MRμTexture transfer function, to enable sensitive and robust tracking of dementia development is outlined here:
First, obtain similarly stained histology of 1) healthy, 2) intermediate pathology (as many points as possible), and 3) highly diseased cortical structure.
Obtain MRμTexture spatial frequency data using in silico application of MRμTexture to acquire data from the 2D histology images of these disease stages.
Vary the healthy data histology incrementally to model disease development, acquiring MRμTexture data (spectra) in silico at multiple disease stages as modelled by the incrementally varied histology images and correlating these spectra with disease stage.
Alternatively, vary the MRμTexture data output spectra and see how these different spectra correlate with histology images of disease progression.
Check that the intermediate in-silico-generated spectra are consistent with the intermediate structure generated by visual interpolation.
   This can be done using as many intermediate points as desired, as there are infinite ways to vary the MRμTexture spectra.

This method enables compilation of a library of MRμTexture output spectra vs. modeled textures. This library data can be obtained by either in silico variation of texture parameters such as tissue texture element size, thickness, spacing, variability, randomness, anisotropy, and tissue contrast of the morphologic elements in various directions within the tissue and correlating these models with MRμTexture data output and/or varying this data output—such as varying the modeled k value vs. intensity spectra to determine the affect this has on the tissue texture obtained from these spectra.

Included in the palette of quantities to vary are the biologic tissue contrast expected in a targeted pathology as well as the response to variation of the VOI dimensions. Development of this library enables looking up spectra and correlating them with specific biologic texture signatures or vice versa.

The above method can also be applied to mapping the MRμTexture signal across an organ. Given a conventional MR reference image—during the same exam, high resolution MRμTexture data can then be acquired anywhere across that image by positioning the VOI wherever desired.

This high-resolution data is then mapped at each VOI location within the organ in which we are attempting to determine disease pathology state. Note that for feature sizes larger than several atomic diameters there is no fundamental resolution limit dictated by MR physics on the MRμTexture measurement.

The effect of the VOI (Volume of Interest/sampling volume) dimensions on the MRμTexture output data/spectrum should also be determined as part of the information informing the library that is developed to correlate tissue features with MRμTexture data output features. The MRμTexture measurement samples textural wavelength vs. intensity along a selected direction anywhere in the anatomy. Just as the number of wavelength repeats sampled along the acquisition axis of the VOI will affect the output spectrum, so to, the cross-section of the VOI, over which the texture is sampled, will affect the output signal.

There are multiple methods applicable to sort out the effect of VOI dimensions.

The first is to acquire data in silico from any relevant tissue/pathology measured by 2-dimensional or 3-dimensional histomorphometry, varying the VOI cross section with each measurement and tracking the variation in the data output from MRμTexture. Correlate the output data spectrum features arising from the MRμTexture measure of the tissue with the cross-section varied continuously.

Another method is to measure the tissue features in each cross-sectional direction to determine if knowledge of the average wavelength in any given direction can be used to predict the effect of changing the VOI dimension in that direction.
Use 3D histology data from different tissue regions and determine in silico the effect of cross section on the measured MRμTexture data.
Use tissue sample blocks that are bracketed on either side by histology slices/images. An example is given in FIG. 6 showing prostate gland histology from either side of a 4 mm thick section through a radical prostatectomy specimen.
Acquire MRμTexture data within the tissue block varying the positioning and the cross section of the VOL. Use in-silico image analysis to yield texture across the histology images obtained from either side of the tissue block.
Change the cross-section and location of the VOI within the slab as MRμTexture data is obtained.
Using the in-silico texture analysis of the histology images as start/end points, vary the texture across the thickness of the tissue slab to see, given specific VOI dimensions and positioning, what texture profile engenders the observed MRμTexture signal.

Varying the profile of texture across the thickness of the slab may take the form of in silico modeling of the tissue texture from using the images as endpoints for the textural variation across the thickness of the slab. This could be a monotonic variation, or it can be estimated as a step change. As a first attempt, it could be estimated that the change in texture size/density is unidirectional between the two images.

Clearly, there are many potential solutions to the problem, but the measured MRμTexture spectrum can be used as the ground truth for determining a possible tissue profile across the thickness of the tissue slab.

Try multiple pathways to morph from one histology image, across the thickness of the slab, into the second image, in-silico and determine which is most apt to yield the observed MRμTexture data spectrum actually acquired in the intervening tissue.

Alternatively, 3D histology methods such as cited previously in the paper by Katsamenis et al. (3DμCT (Katsamenis et al., X-ray Micro-Computed Tomography for Nondestructive Three-Dimensional (3D) X-ray Histology, The American Journal of Pathology, Volume 189, Number 8, August 2019)) would simplify this determination by providing a complete high resolution data set throughout the specimen thickness.

The aim of these modelling methods is to predict the MRμTexture signal without needing hundreds of thousands of histology/image reads, as would be required if the ground truth used was patient status data rather than point by point histologic texture pathology data. Here we will use in silico modelling based on histology to predict the transfer function. Basically, the methods disclosed here are to:
Provide an in-silico tissue morphologic model of each disease tissue state, at various stages of disease, using histology data.
Second, vary this in silico model in its tissue structure and contrast, recognizing that MR contrast may be different than the contrast in a histology or microCT ground truth data set.
Acquire data in silico from each model and correlate with disease stage.
Vary the spectrum to generate intermediate morphologic models/MRμTexture spectra.
Because MRμTexture is a direct measure of structure, with a one-to-one correspondence between signal and texture, in silico modeling enables determination of the texture underlying the MRμTexture signal. Use the correlations developed between modelled texture and structural spectra to inform a library of such correlations.
Correlate the modelled textural morphology with spectra using machine learning—this will enable extrapolation to intermediate tissue textural stages/spectra.
Iteration between each such correlation enables filling out the library to enable clear correlation between textural signature and underlying morphology.

### Machine Learning and Diagnostic Biomarker Extraction:

Development of the biological model is accomplished by correlating tissue texture measurements across the image with disease stage using machine learning techniques.

The difference between diseased and healthy spectra can be used as a metric—i.e. a determination is made of how far off healthy texture is the diseased tissue texture. Alternatively, ratios of spatial wavelength intensity across parts of the spectrum in normal/healthy tissue vs. diseased tissue can be used as the metric. One quantifier for disease would be the difference between the in silico data output from a healthy sample and how far the data must be adjusted in silico to get the MRμTexture data output of another disease stage.

Heterogeneity is a marker of disease also. Correlation of the variation in MRμTexture signal across a lesion with the variation in genetic heterogeneity across the tumor can inform a library of MRμTexture signature vs. genomic signature, similarly to what is done with imaging measurements in Radiogenomics but using the MRμTexture data as one of the comparators.

### Pathology Progression and Changing Chemical Composition of the Textural Elements:

As disease progresses, biologic tissue microtexture changes morphologically and may also change in chemical composition—different chemical components have different resonances. Hence the spectrum of measured signal intensity vs. k-values derived from the MRμTexture measurement changes to reflect this variation in relative quantity of different chemical constituents.

Just as the MRμTexture output data is dependent on what MR contrast is used for data acquisition, in silico acquisition from conventional tissue histopathology will vary depending on the stain used in a particular type of tissue. The stain may highlight different texture constituents, hence changing the derived MRμTexture output spectrum. Part of the power of simulation is the ability to vary tissue contrast, chemical composition, as well as tissue morphology to generate different MRμTexture spectra.

Toward using this ability, histology staining can be chosen to highlight different tissue structures to determine what each textural component contributes to the MRμTexture output data/spectrum—i.e. in cortex, highlight pyramidal neurons, or myelination, or dendrites as all of these structures vary with disease/pathology progression.

One way to do this is via the use of in silico modelling of tissue textures and in silico data acquisition towards correlating individual MRμTexture output spectrum features with the underlying tissue texture. Libraries built up from this correlation between the output spectra obtained from acquiring texture data in silico, and the underlying microtexture known from ground truth data would then enable determination of pathologic tissue texture from spectra acquired in vivo.

These in silico models that are developed and compared to histology images can be used to determine the transfer function for the MRμTexture diagnostic method.

### Data Correlation Towards Biomarker Extraction

Tissue textures are complex, hence determining the transfer function of the MRμTexture method, in order for accurate determination of pathology, is complex. The highly structured data output of MRμTexture is perfect for use of AI algorithms, to identify patterns and correlate the spectrum to identify the underlying structure.

Training of the MRμTexture data output can be accomplished through use of both supervised and unsupervised application of machine learning. In the case of applying in silico tissue modeling and in silico data acquisition, supervised machine learning can be effected through use of ground truth provided by histology, MR-microscopy, patient annotations that include other diagnostic information; unsupervised machine learning is achieved through use of patterns in the MRμTexture in silico output data, these patterns then being associated with diagnostic information obtained via other methods across diseases and disease stages.

### Ground Truth Towards Determination of the Transfer Function

The better the ground truth, the easier it is to determine calibration for the MRμTexture method.

The highest resolution currently is from 2D histology. New technique based on microCT, 3D histology, can get about 20 μm image resolution (9 μm voxel resolution).

Alternatively, MR-microscopy provides the best basis as a 3D ground truth.

Using in silico modeling to determine where spectra change under influence of disease progression. Use a ratio-metric comparing these parts of the spectrum as disease stager.

Unravelling the MRμTexture Signal from Multiple Organs/Multiple Pathologies in Order to Track Disease Progression—Example: COVID-19

Due to the highly complex and varied presentation of pathology in the COVID-19 pandemic, there is immediate need for a high-resolution 3D texture (histologic) diagnostic tool to understand pathology development/disease etiology of this disease for which understanding is still in its infancy.

Doctors and researchers are in dire need of diagnostics that can provide information on the underlying drivers of the disease to help them determine treatments, interventions, and therapies. The paradigm-changing MRμTexture method is unique in its ability to enable tracking of disease progression in COVID-19, a disease for which, unlike most anything the medical community has seen previously, pathology occurs across many organ systems, in highly varied presentations and temporal unfolding, the specific manifestation of disease progression depending on varied interactions between pathology development across multiple organs. Unlike, for example, liver disease, where diagnostic focus is that specific organ, COVID-19 requires diagnostics with the ability to non-invasively track and assess pathology development across a range of organs all implicated in the disease manifestations. What is needed is intra-organ and inter-organ quantitative structured measurements tracking pathology development such as you would be obtained from biopsy-driven histology. The problem here is that biopsy is highly invasive, hence is not a pertinent method from which to obtain diagnostic information across the anatomy in this case, especially in immune-compromised patients. However, MRμTexture can provide the requisite information, as it can be applied in any tissue system for which MR contrast can be developed. And its high-information-content data output can be combined with all other available data sources obtained for a patient. Application of machine learning/deep learning algorithms to the entirety of this diagnostic information can yield correlational data to provide training/calibration for the MRμTexture data output, enabling MRμTexture diagnostic biomarker extraction from the sum of the data sources.

For gauging disease progression in the case of COVID-19 and understanding the underlying pathology drivers, serum markers, CT, and patient workup provide much information, especially as the disease progresses so rapidly that tissue changes can quickly become clearly manifest at this level. However, serum markers at best are indicators of the current rate of disease—not the integrated pathology development and resultant damage. CT and other clinical imaging modalities lack the resolution to measure the microscopic tissue changes, knowledge of which would provide a sensitive, high-information-content measure of pathology development across tissue types, a measure for which there is a dire need. Only MRμTexture can provide this multi-organ microscopic assessment non-invasively. This is especially true given the lack of understanding of this disease and what drives pathology development. What is known is that it is not, as was initially thought, just a respiratory disease. To unravel the disease factors that drive the wide and varied range of presentations and that attack multiple organ systems, leaving a trail of damage, a diagnostic capable of providing high-information-content data across multiple organs is requisite.

Patients present with multiple indications, such as some combination/advancement of kidney damage, poor liver function, blood clots, cardiac inflammation, poor heart function . . . . Along with the need to track pathology through the various organs as the disease progresses, knowledge of the disease force driving this progression is needed.

It has recently been hypothesized (JAMA neurology, Apr. 10, 2020) that the various manifestations of COVID-19 pathology may be driven by underlying neuropathology. With any indication of brain involvement MR becomes a modality of choice-obtaining more information of disease etiology becomes paramount. The brain is clearly an organ from which biopsy-driven histology is not easily obtainable, another reason the histologic-level resolution provided by MRμTexture is a game-changer in ability to assess COVID-19 pathology development.

If the virus attacks the heart and blood/vessels, causing vascular inflammation, leaky vessels, and/or the often observed, pathologic blood clotting, then this neuropathology would be expected to drive other symptoms. And, if indeed one of the primary drivers is neuropathology that affects multiple other organ systems, this can help explain the hugely varied presentations and symptom intensity that is a hallmark of COVID-19. Neuropathology can drive respiratory symptoms, heart trauma, kidney failure, multi-organ distress . . . .

What is needed here is detailed information on correlation between pathology level in the brain and associated pathology level in multiple other organs. If this pathology is well developed you may be able to measure it with CT. But often, the ability to see microscopic changes in organ tissue is required. A macroscopic pathology in one organ (brain for example) can trigger pathology that begins at the finest level of degradation in other organ tissues. MRμTexture is capable of measuring changes in microvasculature as an early harbinger of neuronal involvement. This information can then be correlated with other diagnostic assessments from the same or other organs. Correlation of pathology across organs is enabled by the ability of MRμTexture to measure tissue texture in any organ for which MR contrast can be set.

Further, a high-resolution diagnostic such as MRμTexture is needed to provide detailed measure of patient response to new therapies as they come online. And, on the recovery side, high resolution, sensitive measure of tissue across organs is needed to closely monitor progression/warn of regression.

Is there a clear correlation between brain microvasculature, inflammation, and other symptoms? Or lung vs. brain, and other organs. Are blood clots in the brain mirrored in organ failure elsewhere in the anatomy? Is COVID-19 a neuro-inflammatory condition? These questions can be addressed when adding the high-information content data of MRμTexture to assessments from other diagnostic methods.

MRμTexture provides accurate, high resolution measures of the pathology progression indicated by degradation of biologic tissue textures across patient anatomy. Combined with other measures of pathology from imaging/serum markers/patient presentation across multiple longitudinal measures, a growing body of data informs these correlations enabling a high-resolution, sensitive measure of pathology as MRμTexture fills in the microscopic scale measures of tissue degradation across the anatomy for correlation with macroscopic data. As an increasing amount of patient diagnostic data is acquired for correlation, the accuracy and robustness of MRμTexture's diagnostic capability increases, providing much-needed diagnostic data towards understanding a disease. More than just single organ diagnostic assessment is possible, but also information on organ pathology interactions.

The ability to easily combine the highly structured MRμTexture data with all the other sources of diagnostic data acquired from a patient gives MRμTexture the potential to be developed as a diagnostic of great power in a disease such as COVID-19, where the sources of pathology are so varied.

Towards unraveling the etiology of COVID-19 disease, MRμTexture can be used to:
Measure the pathologic tissue microtexture morphology that develops in multiple organs in response to SARS CoV2 infection, towards understanding disease etiology.
Track the progression of this tissue pathology through multiple organs using both MRμTexture and data from other diagnostics and patient data. Track residual pathology longitudinally over the course of the healing/recovery process-something for which the high-resolution/high-information-content capability of MRμTexture is an enabling method.
Compare underlying tissue microstructure and changes associated with differential disease presentation and progression (normal tissue through pathology development).

The high-resolution capability of the MRμTexture diagnostic provides information on brain pathology, and the ability to track this pathology longitudinally. This information can then be combined with longitudinal information from MRμTexture data from other organs, as well as serum data, CT, x-ray and both structured and unstructured patient annotations, all of which sources will factor in training the MRμTexture toward realizing its potential as a powerful disease diagnostic.

In effect, MRμTexture provides the ability to probe the disease pathology with a high-resolution microscope, and combine the information acquired with all the other sources of information, the various forms of data being combined using machine learning algorithms to identify patterns in the data, yielding biomarker extraction through correlation across the various data sources. This diagnostic capability enables very-high-information content assessment of disease status and progression.

To train the MRμTexture diagnostic to provide the most useful diagnostic information, in silico modelling of the tissue changes attendant with the various targeted symptoms, through use of postmortem histology from the various infected organs is accomplished. This model can be constantly updated with new information on pathology interactions across the various diseased tissues at any given timepoint. By this method, the MRμTexture diagnostic is both providing ongoing information on pathology status and progression and, by ongoing correlation with all other data sources, will be refined and calibrated through diagnostic training provided by this correlation.

Having now described various embodiments of the invention in detail as required by the patent statutes, those skilled in the art will recognize modifications and substitutions to the specific embodiments disclosed herein. Such modifications are within the scope and intent of the present invention as defined in the following claims.

What is claimed is:

1. A method for calibration of the MRμTexture method comprising:
generating a plurality of model datasets representing a continuum of structures with a continuum of biomarker values by morphing data of a 2D structure or 3D structure of a first known disease state to a 2D structure or a 3D structure of a second known disease state; and
applying MRμTexture in silico to extract a simulation data set of texture prevalence for a selected one of a plurality of intermediate morphed conditions corresponding to the plurality of model datasets.

2. The method as defined in claim 1 wherein the step of generating a continuum of structures comprises modeling the structure in silico and morphing the in silico model.

3. The method as defined in claim 1 wherein the step of generating a continuum of structures comprises employing histology as a ground truth for the first known disease state and the second known disease state.

4. The method as defined in claim 1 wherein the 2D structure or 3D structure is a representation of a selected one of bone, liver, prostate, brain, pancreas, or organs in general.

5. The method as defined in claim 1 wherein the step of applying MRμTexture in silico to extract a data set of texture prevalence includes varying the contrast.

6. The method as defined in claim 1 wherein the step of applying MRμTexture in silico to extract a dataset of texture prevalence comprises:
setting a first receiver bandwidth to delineate a length of a VOI;
varying the bandwidth and measuring a mean and range in the datasets to quantify the texture in a segment of a feature size spectrum for a select set of k-values.

7. The method as defined in claim 1 wherein the step of applying MRμTexture in silico to extract a data set of texture prevalence includes preforming a Fourier analysis of the 2D or 3D in silico model and selecting the Fourier coefficients along the axis corresponding to the desired analysis direction in the VOI to provide a Fourier series of k-encoded simulated MRμTexture method signals.

8. The method as defined in claim 7 wherein the step of applying MRμTexture in silico to extract a data set of texture prevalence further includes simulating the signal for a single k-encode by first summing the signal values for all points on the one or two axes (for 2D and 3D models respectively) orthogonal to the analysis direction in the VOI for each point along the analysis direction to generates a 1D signal intensity vs. position array along the analysis direction of the VOI; multiplying the array by a complex sinusoid with a wavelength corresponding to the desired k-encode wherein the complex sum of the points in this product array provides a simulated MRμTexture method signal.

9. The method as defined in claim 1 further comprising:
correlating variation in MRμTexture signal across a structure with variation in genetic heterogeneity within the structure; and,
informing a library of MRμTexture signature vs. genomic signature.
